(19) United States
(12) Patent Application Publication
(10) Pub. No.: US 2011/0282237 A1
(43) Pub. Date: Nov. 17, 2011

(54) TROCAR WITH SPECIMEN RETRIEVAL FEATURE

(75) Inventor: Sean P. Conlon, Loveland, OH (US)

(73) Assignee: ETHICON ENDO-SURGERY, INC., Cincinnati, OH (US)

(21) Appl. No.: 12/780,064

(22) Filed: May 14, 2010

(51) Int. Cl. A61B 10/02

(52) U.S. Cl. 600/562

(57) ABSTRACT

Devices and related methods are provided that generally involve a specimen retrieval pouch coupled to and/or disposed within a surgical access device. Once a specimen is positioned within the pouch, the pouch can be closed and/or contracted to contain the specimen therein, for example by actuating one or more drawstrings configured to contract an open end of the specimen retrieval pouch.

### FIELD

The present invention relates to methods and devices for performing surgical procedures, and in particular to methods and devices for retrieving a specimen during surgery.

### BACKGROUND

It can be desirable during a surgical procedure to retrieve one or more specimens from a surgical site (e.g., from within a body cavity). Retrieval of such specimens can be difficult or even impossible in many procedures however, and particularly in minimally invasive procedures.

In laparoscopic abdominal surgery, for example, a surgical access device can be implanted through an opening in the abdominal wall and the abdominal cavity can be insufflated with pressurized gas. The surgical access device can define one or more working channels through which various laparoscopic instruments can be passed to view, engage, and/or treat tissue. The working channels usually include one or more seals disposed therein to prevent the escape of insufflation gas. When it is desirable to retrieve a specimen in such procedures, laparoscopic graspers are generally used to grasp the specimen and pull it proximally through the working channel in which the graspers are disposed and out of the proximal end of the access device. In such cases, the specimen can be damaged when it is pulled through the seals in the working channel. The seals can also cause the specimen to dislodge from the graspers, which can undesirably lengthen the duration of the surgery and potentially increase the risk of patient complications. Furthermore, only specimens of limited size can be retrieved using such methods, since larger specimens will not fit through the seals or even through the working channel itself. Additionally, it is sometimes desirable to place the specimen within a retrieval bag and then temporarily leave the bag within the body cavity while other surgical activities are performed.

Accordingly, there is a need for improved methods and devices for retrieving a specimen during surgery.

### SUMMARY

The devices and methods disclosed herein generally involve a specimen retrieval pouch that is coupled to and/or disposed within a distal portion of a surgical access device. After inserting a specimen into the pouch, at least a portion of the pouch can be contracted and/or closed to at least partially contain or capture the specimen therein.

In one aspect, a surgical access device is provided that includes a housing having a proximal end and a distal end and at least one port extending therethrough. The at least one port can be configured to receive a surgical instrument therethrough. The device can also include a specimen retrieval pouch coupled to the housing and configured to receive a specimen collected by a surgical instrument inserted through the at least one port and a first drawstring configured to contract a distal perimeter of the pouch.

In one embodiment, the housing can include a retractor coupled to the distal end thereof and the retractor can be configured to be disposed within tissue to form a pathway through the tissue and into a body cavity. In another embodiment, the device can include a second drawstring configured to contract a proximal perimeter of the pouch. The pouch can optionally be substantially cylindrical or substantially conical. The first drawstring can extend from a distal end of the pouch and through a tunnel formed along an inner surface of the housing.

The device can also include a resilient support member coupled to at least a portion of the distal perimeter of the pouch. In one embodiment, the resilient support member can be formed from a shape memory material. The resilient support member can include at least one sleeve configured to couple the first drawstring to the support member. The first drawstring can extend around the entire distal perimeter of the pouch and can include a slipknot tied thereon. In one embodiment, the slipknot is positioned within the housing, adjacent to a bearing surface of the housing configured to bear against the slipknot when the drawstring is translated proximally with respect to the housing.

In another aspect, a specimen retrieval system is provided that includes a surgical access device configured to be inserted through an opening in tissue, a pouch coupled to the surgical access device, and an actuator element extending through a sidewall of the surgical access device and coupled to the pouch such that tension applied to a proximal end of the actuator element is configured to contract a distal perimeter of the pouch.

In one embodiment, the actuator element can be or can include a length of suture extending around the distal perimeter of the pouch and can have at least one slip knot tied thereon. The system can optionally include a support wire that runs alongside the actuator member. In one embodiment, the surgical access device can include a proximal housing having a tissue retractor coupled to a distal end thereof, and the pouch can be disposed within the tissue retractor.

In a further aspect, a method for retrieving a specimen from a surgical site is provided. The method can include inserting an access device having a proximal end and a distal end through an opening in tissue to form a pathway through the tissue and into a body cavity. The method can also include pulling a specimen proximally into a pouch disposed within the access device and actuating a drawstring extending along a sidewall of the access device to contract a distal circumference of the pouch to retain at least a portion of the specimen within the pouch.

The method can further include removing the access device and the specimen from the opening. In one embodiment, pulling the specimen proximally can include grasping the specimen with an instrument passed through the access device and retracting the instrument proximally with respect to the access device. The method can also include removing a support member from the access device before contracting the distal circumference of the pouch and/or removing a housing portion of the access device having at least one seal disposed therein and then removing the specimen through the opening. In one embodiment, actuating the drawstring comprises pulling a terminal end of the drawstring proximally with respect to the access device.

### BRIEF DESCRIPTION OF THE DRAWINGS

The invention will be more fully understood from the following detailed description taken in conjunction with the accompanying drawings, in which:

FIG. 1 is a perspective view of a surgical access device according to one embodiment of the present invention;

FIG. 2 is another perspective view of the surgical access device of FIG. 1;

FIG. 3 is a side view of the surgical access device of FIGS. 1-2 with a retractor ring of the device shown in phantom;

FIG. 4 is a perspective view of a drawstring according to one embodiment of the present invention;

FIG. 5 is a perspective view of a support member according to one embodiment of the present invention;

FIG. 6 is a perspective view of the drawstring of FIG. 4 and the support member of FIG. 5 positioned adjacent to one another;

FIG. 7 is a side view of the surgical access device of FIGS. 1-3 with various components removed for clarity;

FIG. 8 is a perspective view of the surgical access device of FIGS. 1-3 with various components removed for clarity;

FIG. 9 is a side view of the surgical access device of FIGS. 1-3 with various components removed for clarity and with a retractor ring of the device shown in phantom;

FIG. 10 is a side view of an outer housing of the surgical access device of FIGS. 1-3;

FIG. 11 is a perspective view of the outer housing of FIG. 10 and the drawstring of FIG. 4;

FIG. 12 is a side view of the outer housing of FIG. 10, the drawstring of FIG. 4, and the support member of FIG. 5;

FIG. 13 is a side view of the surgical access device of FIGS. 1-3 shown with the support member removed;

FIG. 14 is a side view of the surgical access device of FIGS. 1-3 shown with the support member removed and with the specimen retrieval pouch in a partially closed configuration;

FIG. 15 is a bottom view of the surgical access device of FIGS. 1-3 shown with the specimen retrieval pouch in an open configuration;

FIG. 16 is a bottom view of the surgical access device of FIGS. 1-3 shown with the specimen retrieval pouch in a partially closed configuration;

FIG. 17 is a side view of a surgical access device according to another embodiment of the present invention;

FIG. 18 is a side view of the surgical access device of FIG. 17 shown with a distal portion of the specimen retrieval pouch in a partially closed configuration;

FIG. 19 is a side view of a surgical access device according to another embodiment of the present invention;

FIG. 20 illustrates a surgical access device implanted in a tissue wall in accordance with a method of retrieving a specimen according to one embodiment of the present invention;

FIG. 21 illustrates the surgical access device of FIG. 20 with a specimen disposed within the pouch;

FIG. 22 illustrates the surgical access device of FIGS. 20-21 with the support member removed;

FIG. 23 illustrates the surgical access device of FIGS. 20-22 with the distal perimeter of the pouch substantially closed around a specimen;

FIG. 24 illustrates a surgical access device implanted in a tissue wall in accordance with a method of retrieving a specimen according to another embodiment of the present invention;

FIG. 25 illustrates the surgical access device of FIG. 24 with a specimen disposed within the pouch;

FIG. 26 illustrates the surgical access device of FIGS. 24-25 with the distal perimeter of the pouch substantially closed around a specimen;

FIG. 27 illustrates the surgical access device of FIGS. 24-26 with the proximal perimeter of the pouch substantially closed around a specimen; and

FIG. 28 illustrates the surgical access device of FIGS. 24-27 with the pouch separated from the access device.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

In general, surgical access devices and methods are provided that involve a specimen retrieval pouch that is coupled to and/or disposed within a distal portion of the access device. A person skilled in the art will appreciate that, while methods and devices are described herein in connection with minimally invasive laparoscopic procedures in the abdominal cavity, the methods and devices can be used in almost any part of a human or animal body and in various other types of surgical procedures. By way of non-limiting example, the devices and methods disclosed herein can be used in the thoracic cavity, pelvic cavity, cranial cavity and/or any of the body's natural orifices and can be used in endoscopic procedures and/or in open surgical procedures.

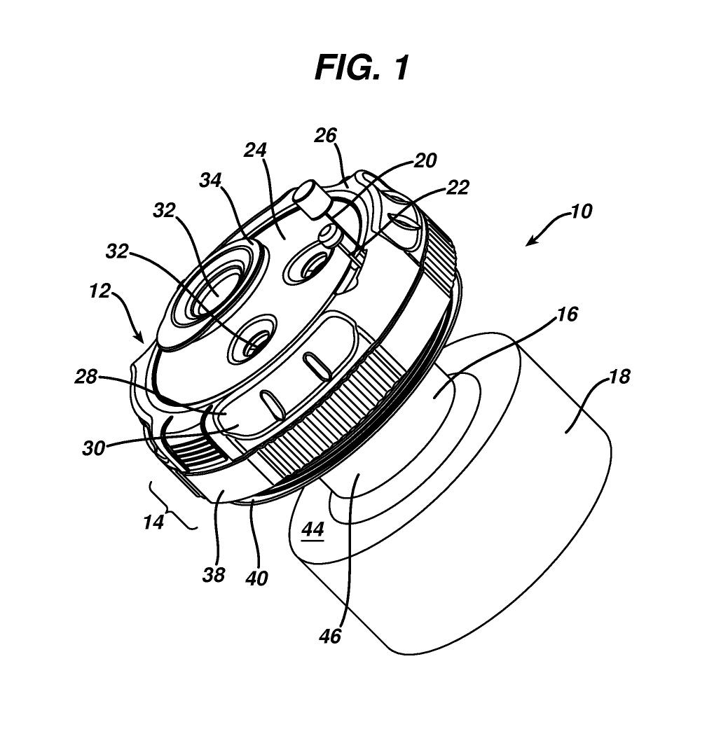

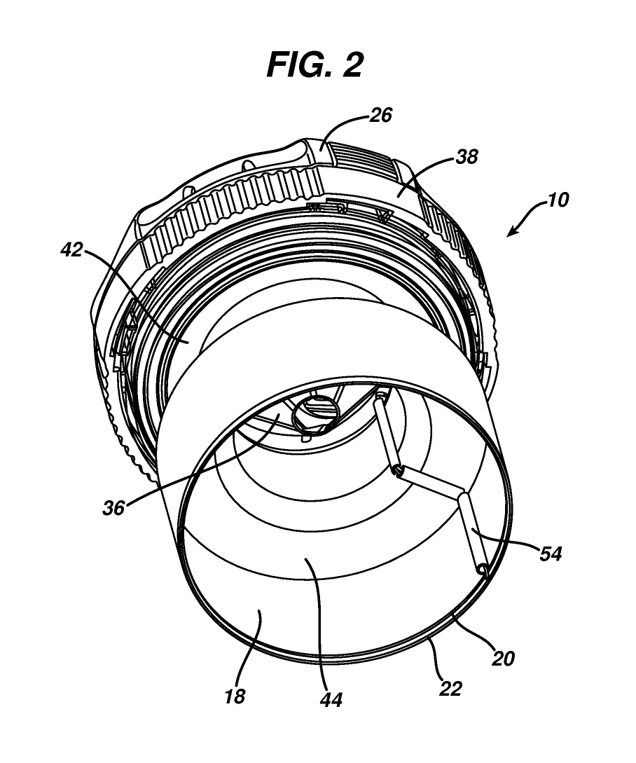

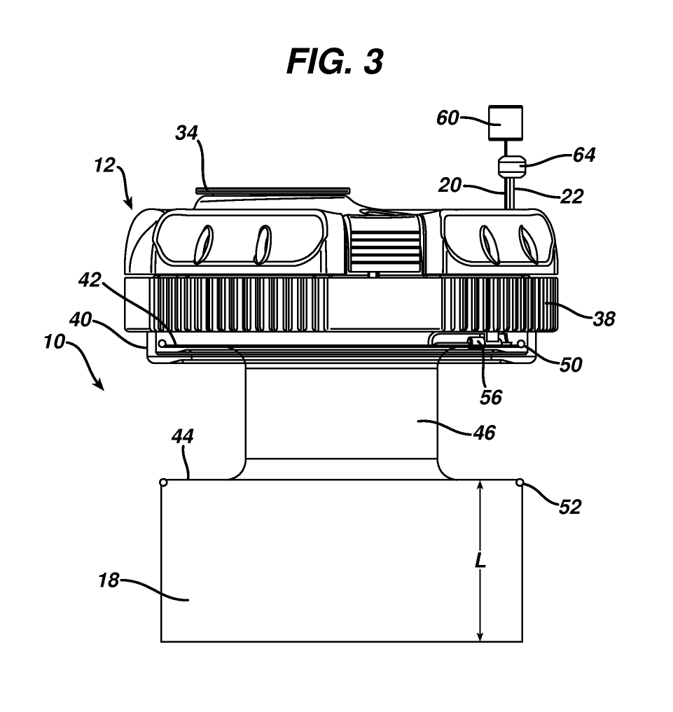

FIGS. 1-3 illustrate one exemplary embodiment of a surgical access device 10. A person skilled in the art will appreciate that the specimen retrieval pouch can be incorporated into a variety of access devices, including trocars and cannulas, and that the access device 10 is merely one example of such an access device. As shown, the device 10 generally includes a housing 12, a ring assembly 14, a retractor 16, and a specimen retrieval pouch 18. The retractor 16 can be configured to be inserted through an opening in tissue, for example through a surgical incision formed in an abdominal wall. Before or after inserting the retractor 16 into a surgical opening, the housing 12 can be selectively coupled thereto via the ring assembly 14. The specimen retrieval pouch 18 can be coupled to and/or disposed within any portion of the device 10, such as the housing 12, the ring assembly 14, and/or the retractor 16. In the illustrated embodiment, the pouch 18 is generally in the form of a distal extension of the retractor 16. Alternatively, or in addition, part or all of the pouch 18 can be disposed within the retractor 16 such that it generally lines the inner surface thereof, as explained in more detail below. One or more drawstrings 20 and/or one or more support members 22 can be provided for manipulating the pouch 18 and/or the retractor 16, such as for contracting and/or closing a distal circumference or perimeter of the pouch 18 to capture a specimen therein.

The housing 12 have a variety of configurations, but in the illustrated embodiment it is generally in the form of a substantially rigid cylindrical body that includes an inner housing 24 and an outer housing 26. The inner housing 24 can be removable from the outer housing 26 or can be formed integrally therewith. The inner housing 24 can optionally be rotatable with respect to the outer housing 26, e.g., rotatable 360 degrees in either a clockwise or counterclockwise direction relative thereto. For example, in the illustrated embodiment, the outer housing includes upper and lower halves 28, 30 which together sandwich the inner housing 24 and form a rotation channel in which the disk-shaped inner housing 24 can be rotatably seated. The upper and lower halves 28, 30 of the outer housing can be selectively coupled to each other, for example via a snap-fit engagement, a threaded coupling, or any other coupling mechanism known in the art. One or more sealing members (e.g., O-rings) can be positioned between the outer and inner housings 24, 26 to form a seal therebetween and to prevent escape of insufflation fluid.

The inner housing 24 can include one or more instrument ports 32 formed therein, each port defining a working channel through which one or more instruments can be passed and/or inserted. A person skilled in the art will appreciate that there can be any number of instrument ports 32 formed in the inner housing 24 and that the instrument ports 32 can have varying sizes, angular orientations, or arrangements with respect to the inner housing 24.

Typically, during surgical procedures in a body cavity such as the abdomen, insufflation fluid (e.g., carbon dioxide gas) is provided through the surgical access device 10 to expand the body cavity to facilitate the surgical procedure. Thus, in order to maintain insufflation within the body cavity, the device 10, and in particular the ports 32, can include one or more seals disposed therein and configured to form a seal thereacross. These seals can prevent insufflation fluid from escaping when surgical instruments are inserted through the access device 10. Various sealing elements are known in the art, but typically the access device 10 can include at least one instrument seal that forms a seal around an instrument disposed through a port 32. The device 10 can also include at least one channel seal or zero-closure seal that seals the instrument port 32 when no instrument is disposed therethrough. Alternatively, or in addition, the device can include a combination instrument seal and channel seal that is effective to both form a seal around an instrument disposed through the port 32 and to form a seal in the port 32 when no instrument is disposed therethrough. A person skilled in the art will appreciate that various seals can be used including, without limitation, duckbill seals, cone seals, flapper valves, gel seals, diaphragm seals, non-linear sealing elements such as sealing elements with an S-shaped opening, lip seals, iris seals, and/or any other type of seal known in the art. A person skilled in the art will also appreciate that any combination of seals can be included in any of the embodiments described herein.

The inner housing 24 can also include at least one safety shield 36 extending from a distal surface of the inner housing 24 into the retractor 16 and/or the pouch 18. The safety shield 36 can help protect the retractor 16 and/or the pouch 18 from being damaged by instruments passed through the access device 10. In the illustrated embodiment, the safety shield 36 is generally in the form of a flexible cone formed from a plurality of layered flaps. The safety shield 36 can be formed from a variety of materials, including for example polyurethane, silicone, urethane, thermoplastic elastomer, rubber, polyolefins, polyesters, nylons, fluoropolymers, any other suitable material known in the art, and/or any combination thereof.

An insufflation port (not shown) can also be provided in the inner housing 24 and/or the outer housing 26 to apply insufflation fluid to a surgical site or to remove such fluid therefrom. The housing 12 can also be provided with one or more reducer caps 34 configured to effectively reduce the diameter of a port 32 in which they are installed. The reducer caps 34 can be selectively coupled to any of the sealing ports 32, for example by a snap-fit connection.

The housing can have a variety of sizes depending on the type of procedure being performed and the quantity and size of the requisite surgical instruments or specimens to be retrieved. In one exemplary embodiment, the housing 12 can have a diameter of about 75 mm, and can include instrument ports having diameters in the range of about 6 mm to about 16 mm. The housing 12 can be formed from a variety of materials known in the art, including but not limited to various polymers, including polycarbonates and polyetheretherketone (PEEK), metals such as titanium or stainless steel, composites such as carbon-fiber reinforced PEEK, various ceramic materials, and/or any combination thereof. The housing 12 can also be formed of various semi-rigid/flexible materials, including polyurethanes such as Pellethane (available from The Dow Chemical Company of Midland, Mich., USA), thermoplastic elastomers such as Santoprene (available from ExxonMobil Chemical of Houston, Tex., USA), polyisoprene elastomers, medium to high durometer silicone elastomers, and/or any combination thereof.

The housing 12 can be directly coupled to the retractor 16, can be formed integrally therewith, or can be coupled thereto via one or more intermediate structures. In the illustrated embodiment, a ring assembly 14 is provided between the housing 12 and the retractor 16 and is configured to selectively couple and seal the housing 12 to the retractor 16. The ring assembly 14 can have a variety of sizes, shapes, and configurations. In the illustrated embodiment, the ring assembly 14 can include a locking ring 38 and a retractor ring 40 which can each be in the form of a substantially circular ring. Alternatively, the ring assembly 14 can include a single, integrally-formed ring. In the illustrated embodiment, the locking ring 38 and the retractor ring 40 can be selectively and/or releasably mated to each other and to the housing 12 and the retractor 16. For example, the housing 12 can be selectively coupled to the locking ring 38 using any of a variety of engagement and/or release mechanisms, including without limitation a snap-lock mechanism, a bayonet coupling, a threaded interface, a friction fit, and/or one or more screws, pins, nails or other fasteners. For example, the housing 12 can include one or more bayonet pins (not shown) arranged radially about an outer circumference of the housing 12 (e.g., extending distally from the lower surface of the outer housing). The bayonet pins can be configured to engage corresponding slots formed in an inner circumferential surface of the locking ring 38. Rotation of the outer housing 26 in a first direction with respect to the locking ring 38 can cause the pins to slide along laterally extending portions of the slots until they snap into a terminal end thereof, thereby locking the housing 12 to the locking ring 38. Rotation of the outer housing 26 in a second, opposite direction can cause the pins to slide back along the laterally-extending portion of the slots to a vertically-extending portion, at which point the pins can be withdrawn proximally from the slots and the outer housing 26 can be separated from the locking ring 38. One or more locking mechanisms can also be provided to selectively prevent rotation of the outer housing 26 with respect to the locking ring 38. For example, a depressible tab can be provided that is configured to allow rotation of the outer housing 26 only when the tab is depressed by a user.

As noted above, the locking ring 38 can be coupled to a retractor ring 40, which can in turn be coupled to the retractor 16. Any of the methods described above for attaching the inner housing 24 to the outer housing 26, the upper half 30 of the outer housing to the lower half 28 of the outer housing, and/or the outer housing 24 to the locking ring 38 can be employed to couple the locking ring 38 to the retractor ring 40. For example, the locking ring 38 and the retractor ring 40 can have corresponding threaded interfaces, or can have corresponding pins and slots. To mate the ring assembly 14 and the retractor 16, a proximal flange 42 formed at the proximal end of the retractor 16 can be folded or bent to fit through a central opening in the retractor ring 40 having a diameter that is less than that of the proximal retractor flange 42. Once advanced through the central opening, the proximal retractor flange 42 can be unfolded such that it is positioned on an inner circumferential lip of the retractor ring 40. The proximal flange 42 can thus be disposed within the retractor ring 40 to couple the retractor 16 and the ring assembly 14 together. In other words, the proximal retractor flange 42 can generally be clamped or sandwiched between the retractor ring 40 and the locking ring 38. An adhesive can optionally be provided to seal the retractor 16 to the ring assembly 14.

The retractor 16 can thus be configured to extend distally from the housing 12 and/or the ring assembly 14, providing a pathway through tissue into a body cavity (e.g., when the retractor 16 is positioned in an opening formed in tissue). In the embodiment shown, the retractor 16 is flexible and has a proximal flange 42 and a distal flange 44 with an inner elongate portion 46 extending therebetween. The specimen retrieval pouch 18 can extend distally from the distal flange 44 to form a skirt extending from the retractor 16. The inner elongate portion 46 of the retractor 16 can have a diameter less than a diameter of the proximal and distal flanges 42, 44, which can each have the same diameter or can have different diameters from one another. The proximal flange 42 can be configured to be seated within the retractor ring 40, as explained above. Alternatively, or in addition, the retractor 16 can be attached to the retractor ring 40 and/or the housing 12 using an adhesive, sealant, complementary threads, or virtually any other attachment mechanism.

The retractor 16 can include a proximal O-ring 50 positioned within the proximal flange 42 to help provide structural support to the retractor 16 within the retractor ring 40. A distal O-ring 52 can also be provided and can be positioned within the distal flange 44 to provide structural support to the retractor 16 within a patient's body. The proximal and distal O-rings 50, 52 in certain embodiments are substantially flexible, but one or both can be substantially flexible or substantially rigid as needed for use in a particular application.

As discussed below, the retractor 16 can also include at least one support member 22 positioned around a distal circumference or rim of the pouch 18 to provide structural support thereto. In one embodiment, the pouch 18 of the retractor can be gathered or rolled up prior to insertion of the access device 10 into a tissue opening. Once inserted, and at any time during or after a surgery, the pouch 18 can be unfurled, unrolled, or otherwise released, for example via one or more slip knots or by severing one or more temporary restraints. This can advantageously permit the use of a relatively large pouch without interfering with insertion of the access device 10 into a tissue wall.

The retractor can be formed of any suitable material known in the art, including silicone, urethane, thermoplastic elastomer, rubber, and/or any combination thereof. The retractor can also have a variety of other shapes and sizes, and in one embodiment can have a conical or frusto-conical configuration.

The access devices disclosed herein can also include various other features, as disclosed in commonly-assigned U.S. patent application Ser. No. 12/512,542 entitled “Methods And Devices For Providing Access Into A Body Cavity” filed Jul. 30, 2009, the entirety of which is hereby incorporated by reference. A variety of other features, including various release and/or locking mechanisms for selectively attaching and detaching the various components of the access device 10 can also be included without departing from the scope of the present invention. Moreover, other exemplary access devices are described in U.S. Pat. No. 5,330,437 entitled “Self Sealing Flexible Elastomeric Valve and Trocar Assembly for Incorporating Same” issued Jul. 19, 1994; U.S. Pat. No. 7,615,005 entitled “Medical Apparatus For Use With An Endoscope” issued Nov. 10, 2009; U.S. Patent Publication No. 2004/0230161 entitled “Trocar Seal Assembly” filed Mar. 31, 2004; U.S. Patent Publication No. 2006/0247673 entitled “Multi-port Laparoscopic Access Device” filed Apr. 5, 2006; U.S. Patent Publication No. 2007/0185453 entitled “Conical Trocar Seal” filed Oct. 15, 2003; U.S. Patent Publication No. 2009/0005799 entitled “Duckbill Seal with Fluid Drainage Feature” filed Jun. 29, 2007; U.S. Patent Publication No. 2010/0081863 entitled “Methods and Devices for Performing Gastrectomies and Gastroplasties” filed Sep. 30, 2008; U.S. Patent Publication No. 2010/0081864 entitled “Methods and Devices for Performing Gastrectomies and Gastroplasties” filed Sep. 30, 2008; U.S. Patent Publication No. 2010/0081880 entitled “Surgical Access Device” filed Sep. 30, 2008; U.S. Patent Publication No. 2010/0081881 entitled “Surgical Access Device with Protective Element” filed Sep. 30, 2008; U.S. Patent Publication No. 2010/0081882 entitled “Multiple Port Surgical Access Device” filed Sep. 30, 2008; U.S. Patent Publication No. 2010/0081883 entitled “Methods and Devices for Performing Gastroplasties Using a Multiple Port Access Device” filed Sep. 30, 2008; U.S. Patent Publication No. 2010/0081995 entitled “Variable Surgical Access Device” filed Sept. 30, 2008; U.S. patent application Ser. No. 12/399,473 entitled “Methods And Devices For Providing Access Into A Body Cavity” filed Mar. 6, 2009; U.S. patent application Ser. No. 12/399,482 entitled “Methods And Devices For Providing Access Into A Body Cavity” filed Mar. 6, 2009; U.S. patent application Ser. No. 12/399,547 entitled “Surgical Access Devices And Methods Providing Seal Movement In Predefined Paths” filed Mar. 6, 2009; U.S. patent application Ser. No. 12/399,625 entitled “Methods And Devices For Providing Access Into A Body Cavity” filed Mar. 6, 2009; U.S. patent application Ser. No. 12/399,633 entitled “Methods And Devices For Providing Access Into A Body Cavity” filed Mar. 6, 2009; U.S. patent application Ser. No. 12/399,656 entitled “Surgical Access Devices And Methods Providing Seal Movement In Predefined Movement Regions” filed Mar. 6, 2009; U.S. patent application Ser. No. 12/512,568 entitled “Methods And Devices For Providing Access Into A Body Cavity” filed Jul. 30, 2009; and U.S. patent application Ser. No. 12/766,086 entitled “Methods And Devices For Accessing A Body Cavity” filed Apr. 23, 2010, all of which are incorporated by reference herein in their entireties. Any of these access devices can be used in combination with a specimen retrieval pouch.

As indicated above, the device 10 can also include at least one specimen retrieval pouch 18. The pouch can be formed from a variety of materials, and can generally be formed from a material that is flexible, stretchable, deformable, and/or resistant to tearing and puncture. For example, the pouch can be formed from polyurethane, silicone, thermoplastic elastomer, rubber, polyolefins, polyesters, nylons, fluoropolymers, any other suitable material known in the art, and/or any combination thereof. In an exemplary embodiment, the pouch 18 can be formed from a polyurethane film having a thickness of approximately 0.05 mm. In the illustrated embodiment, the pouch is in the form of a skirt extending from the retractor 16, although as explained in more detail below, the pouch can also be in the form of a liner for the retractor 16 and/or the pouch can be detachable from the device 10 as needed in a particular procedure.

The pouch 18 can have a variety of shapes and sizes, depending on the specimen or specimens to be retrieved. In one embodiment, the pouch 18 can have a diameter substantially equal to that of the distal O-ring 52 of the retractor 16, for example 65 mm, and it can have a length L of about 70 mm. The pouch 18 can also be considerably larger or smaller than the retractor 16. The pouch 18 can be formed integrally with the retractor 16 as shown, or can optionally be mated thereto using any of a variety of attachment mechanisms, e.g., adhesives, screws, press fittings, and the like. The pouch can also have a conical, frusto-conical, or any other shape configured to capture a specimen therein.

The access device 10 can also include one or more drawstrings or actuator elements configured to effect closure of the pouch 18 or to otherwise manipulate the pouch 18. In the illustrated embodiment, a drawstring 20 can be inserted and/or threaded through a channel formed in a distal circumference or perimeter of the pouch 18. The opposed ends of the drawstring 20 can be threaded through a protective tunnel 54 formed in or coupled to an inner wall of the retractor 16 and/or the pouch 18. Thus, the two ends of the drawstring 20 can be threaded proximally along an inner wall of the pouch 18, and can then make a series of bends (e.g., ninety degree bends) such that they run radially inward along a distal-most surface of the distal retractor flange 44, proximally along an inner surface of the inner elongate retractor portion 46, and radially outward along a proximal-most surface of the proximal retractor flange 42. As the two lengths of the drawstring 20 run along the surface of the proximal flange 42, or at virtually any other point along their length, they can be tied together in a slipknot 56. A single strand of the drawstring 20 can then extend away from the slipknot, proximally through the locking ring 38 and the outer housing 26, and then can ultimately terminate in a pull handle 60 configured to be grasped and pulled by a user. The pull handle 60 can optionally be in the form of a pull ring, a finger loop, and/or a reel mechanism and it can be selectively coupled to the drawstring 20 (e.g., to facilitate threading of the drawstring 20 through the housing 12 or other components of the access device 10 having an opening smaller than a cross-section of the pull handle 60).

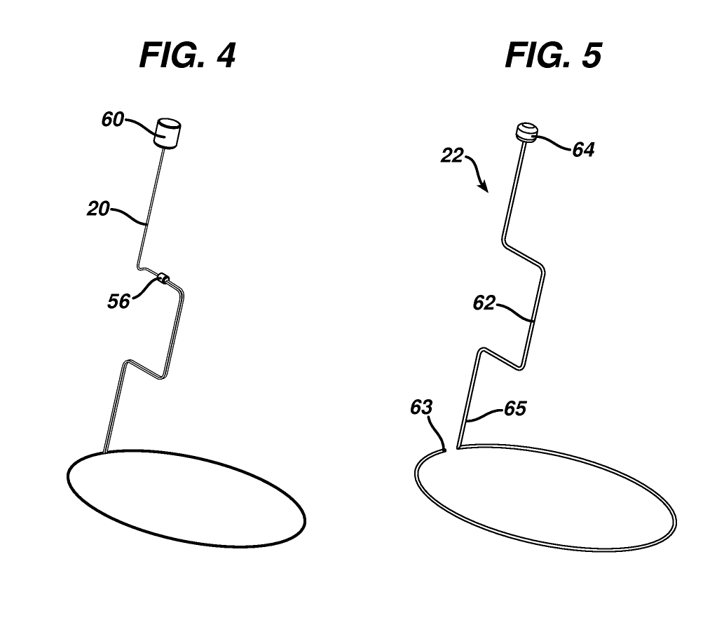

The drawstring 20 can be substantially flexible and it can be formed from a variety of materials known in the art to be suitable for surgical applications and capable of withstanding moderate tension forces, such as a suture thread. The drawstring 20 can optionally be coated with a friction-reducing material to facilitate sliding of the slip knot 56 along the drawstring 20 and/or sliding of the drawstring 20 within the pouch 18. FIG. 4 illustrates the drawstring 20 without the other portions of the access device 10 being shown in order to more clearly illustrate the various bends formed therein. The slip knot 56 and pull handle 60 are also illustrated.

The access device 10 can also include one or more support members or support wires 22 configured to provide structural rigidity to the pouch 18 and/or to the retractor 16 itself. As shown in the illustrated embodiment, and particularly in FIG. 5, a single support member 22 is provided that is generally in the form of a wire body 62 and a pull handle 64. The wire body 62 can be formed from a shape memory material, such as Nitinol, such that it can be substantially deformed yet still resiliently return to a pre-determined shape and configuration. In other words, the support member 22 can be heat treated or otherwise configured to resiliently maintain the shape illustrated in FIG. 5, even when it is removed from the device 10. The proximal pull handle 64 can allow a user to grasp and pull the support member 22 out of the access device 10, for example with the access device 10 implanted in a tissue wall. Like the pull handle 60 described above, the pull handle 64 of the support member 22 can be selectively coupled to the support member 22 such that it can be removed when threading the proximal end of the support member 22 through small openings (e.g., through the housing 12 or other components of the access device 10). To facilitate removal of the support member 22 from the access device 10, the distal end 63 of the wire body 62 can extend around less than 360 degrees of the distal circumference of the pouch 18 such that it terminates a distance apart from the portion 65 of the wire body 62 that extends proximally along a sidewall of the pouch 18. Thus, a user can pull the support member 22, for example using the pull handle 64, proximally out of the access device 10 without rupturing or tearing the pouch 18 and without substantially changing the position of the pouch 18.

In some embodiments, the access device can be initially inserted though an opening in tissue with the support member removed. For example, in embodiments in which the pouch is initially rolled or gathered during insertion of the access device, the support member can be inserted after the access device is positioned in the tissue opening and it can be used to unfurl and/or unroll the pouch.

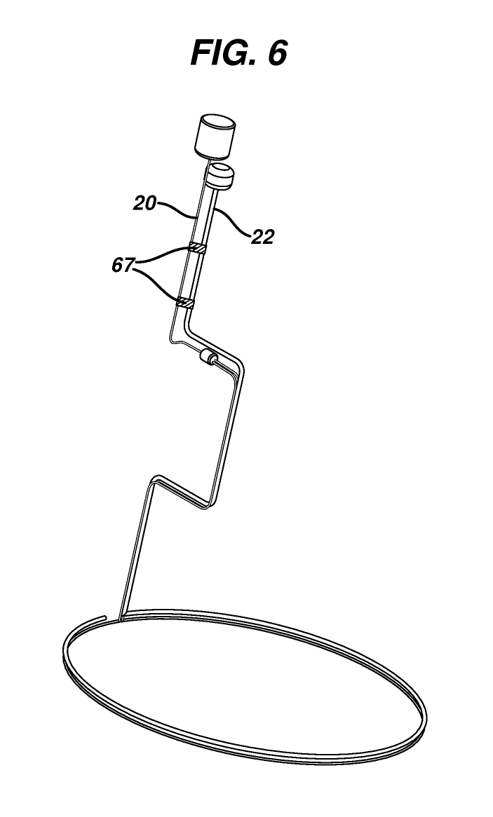

As shown in FIG. 6, the support member 22 can run alongside the drawstring 20, traversing substantially the same path through the access device 10 as the drawstring 20. The support member 22 can thus optionally include one or more sleeves, bands, ties, clips, or other guides 67 positioned incrementally along its length to slidably couple it to the drawstring 20 and thereby help maintain the drawstring 20 in the desired position, for example even while pulling forces are applied to the drawstring 20. In addition, the guides 67 can support the drawstring 20 as it is threaded through the housing 12, for example during initial manufacture of the device or in embodiments in which the device 10 is assembled during the surgical procedure, as discussed further below.

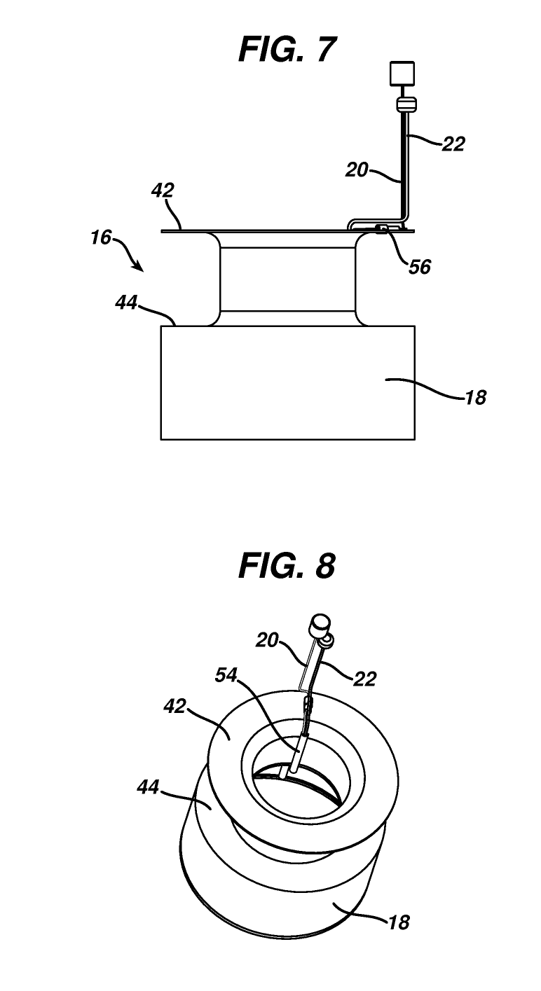

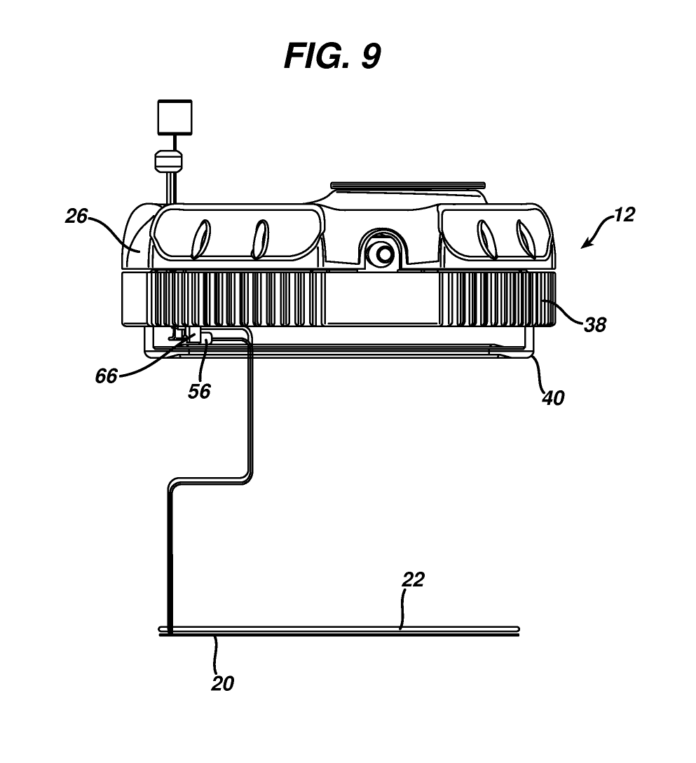

FIGS. 7-9 illustrate the positioning of the drawstring 20 and the support member 22 within the access device 10, with various portions of the access device 10 removed for clarity. For example, FIGS. 7 and 8 illustrate the drawstring 20 and the support member 22 running alongside one another through the protective tunnel 54 of the retractor 16. FIG. 9 illustrates the drawstring 20 and the support member 22 running through the housing 12. Although the drawstring 20 and support member 22 are generally shown exiting the device 10 at a proximal surface of the housing 12, one or both can also optionally exit the device in a sidewall of the housing 12, through the ring assembly 14, and/or through the retractor 16. As shown, the slip knot 56 tied in the drawstring 20 can be positioned inside of the retractor ring 40, adjacent to a boss 66 formed on a distal surface of the housing 12.

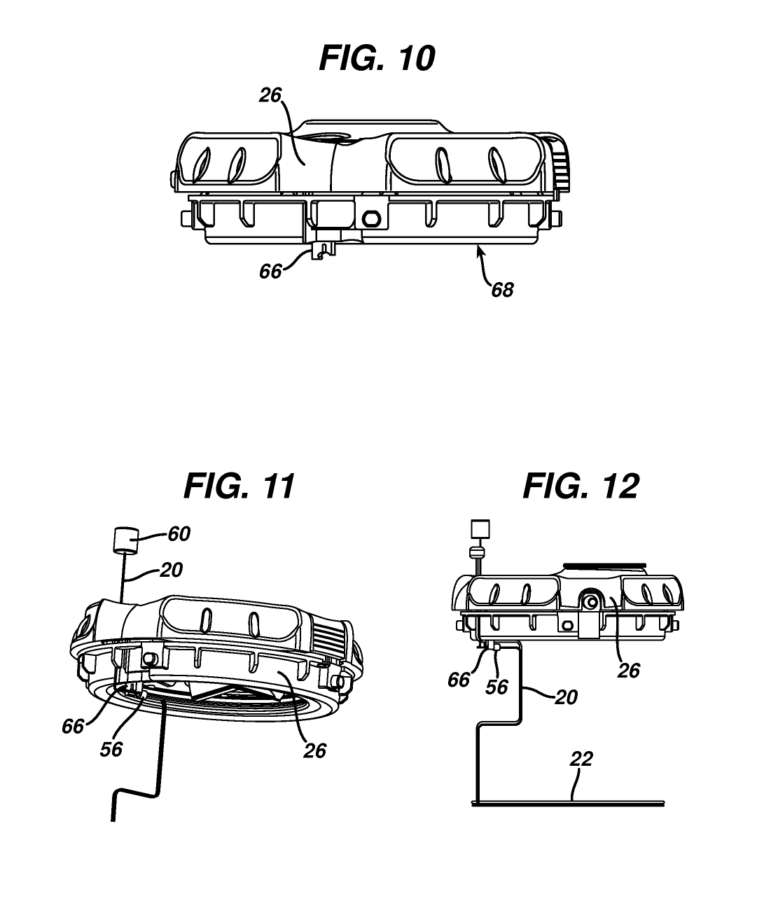

FIG. 10 illustrates a close-up view of the outer housing 26 with other portions of the access device 10 removed for clarity. As shown, the outer housing 26 can include a boss 66 extending distally from a distal surface 68 thereof. The boss 66 can be configured to receive and/or bear against one or more slip knots tied in one or more of the drawstrings. As shown in FIGS. 11-12, the boss 66 can thus provide a stop and/or bearing surface to prevent proximal movement of the slip knot 56 when the drawstring 20 is pulled proximally by the pull handle 60. While the boss 66 is shown as being formed integrally with the outer housing 26, it can also be a separate component attached thereto, or attached to any other appropriate component of the access device 10.

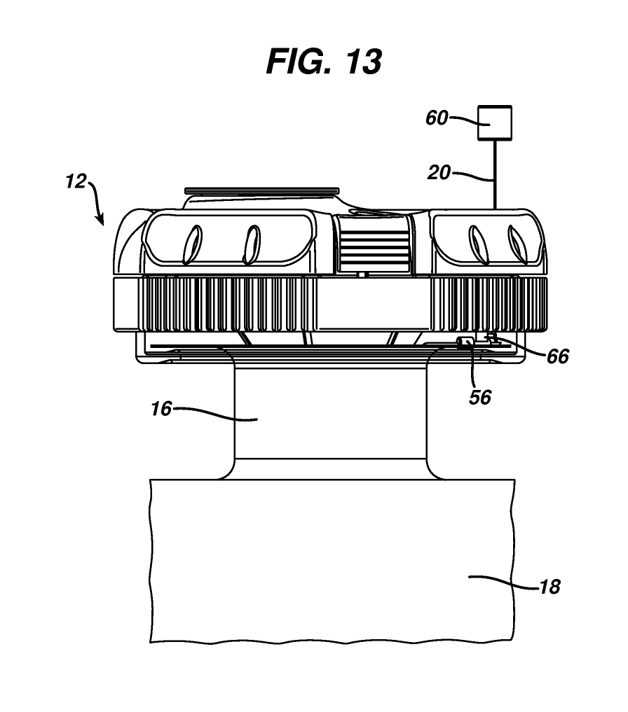

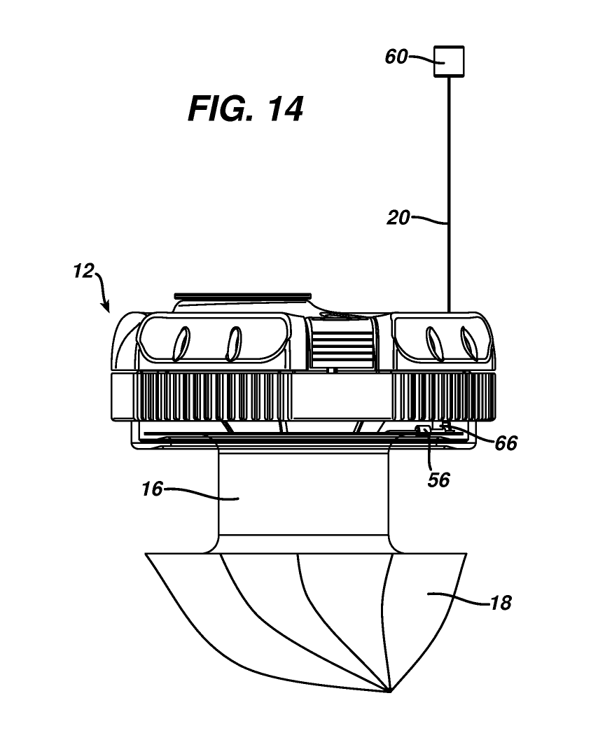

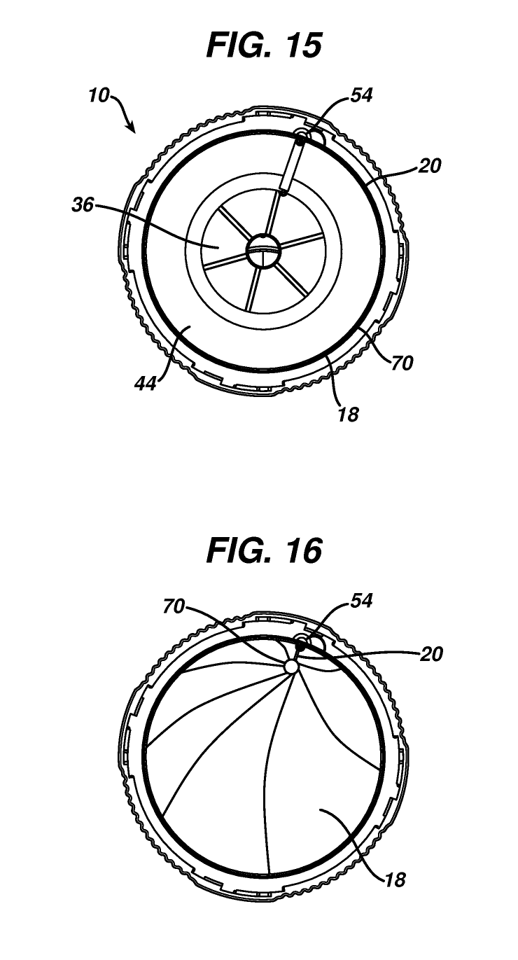

In use, as shown for example in FIGS. 13-16, the support member 22 and the drawstring 20 can be manipulated by a user to effect closure of the specimen retrieval pouch 18. FIG. 13 illustrates the access device 10 after the support member has been removed, for example after a user grasps the pull handle thereof and pulls the deformable support member proximally out of the access device 10. As shown, the walls and distal circumference or perimeter of the pouch 18 lose some of their structural rigidity when the support member is removed. After the support member 22 is removed, or while the support member 22 is being removed, the drawstring 20 can be pulled proximally by the pull handle 60, as shown for example in FIG. 14. As the end of the drawstring 20 to which the pull handle is attached is pulled proximally, one of the strands of the drawstring 20 is pulled through the slip knot 56, which is prevented from moving proximally by the boss 66 of the housing 12. In other words, as the drawstring 20 is pulled, the diameter of a noose-like portion thereof contracts, cinches, and/or closes. The degree of closure can be commensurate with the degree to which the drawstring 20 is pulled (e.g., the length along which the drawstring 20 is pulled). As shown in FIG. 14, the drawstring 20 can be pulled proximally until the distal circumference or perimeter of the pouch 18 is substantially closed. In one embodiment, the inner surface of the distal rim of the pouch 18 can be provided with a layer or bead of adhesive sealant such that a substantially fluid-tight (e.g., air-tight) seal can be formed between the opposing cinched surfaces of the pouch when the pouch is fully closed. FIG. 15 illustrates the access device 10 from below before actuating the drawstring 20. FIG. 16 illustrates the same view of the access device 10, after the drawstring 20 has been actuated to significantly reduce, close, and/or cinch the distal circumference 70 of the pouch 18.

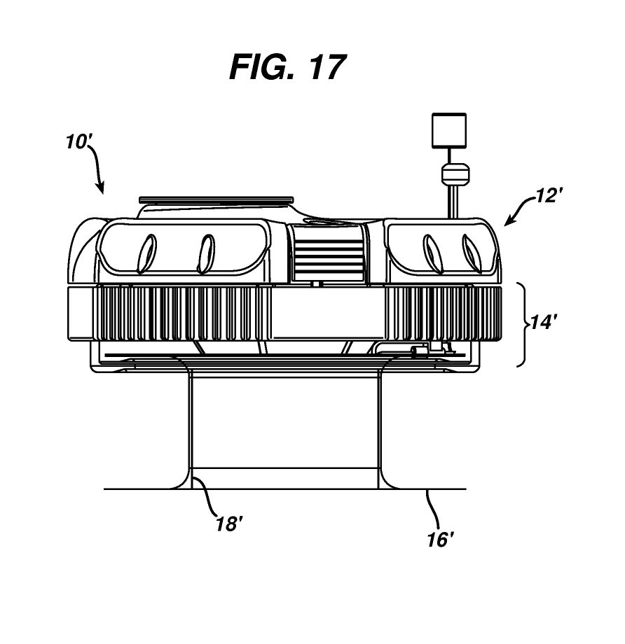

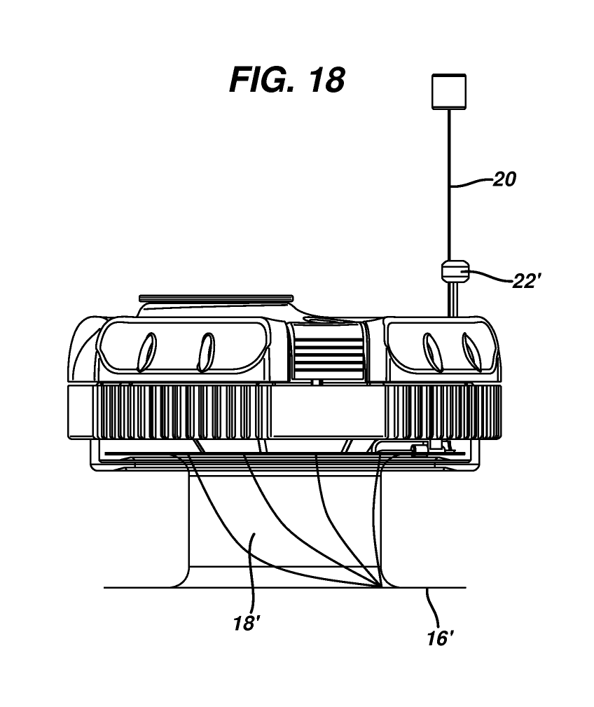

FIGS. 17-18 illustrate another embodiment of an access device 10′ in which the pouch 18′ is separate from the retractor 16′ and generally lines an inner surface thereof. For example, the pouch 18′ can have a shape substantially similar to that of the retractor 16′ and can be coupled at its proximal end to a distal surface of the housing 12′, to a surface of the ring assembly 14′, and/or to a surface of the retractor 16′. As shown in FIG. 18, a drawstring 20′ can be pulled proximally to cinch and/or close a distal circumference or perimeter of the pouch 18′. As shown, the pouch 18′ can be closed even though the support member 22′ remains in place, providing support for the retractor 16′. Alternatively, the retractor 16′ can be substantially rigid or substantially semi-rigid, in which case the support member 22′ can be omitted. In one embodiment, part or all of the pouch 18′ can be adhered or otherwise affixed to the inner surface of the retractor 16′ until separated therefrom by actuation of the drawstring(s) 20′.

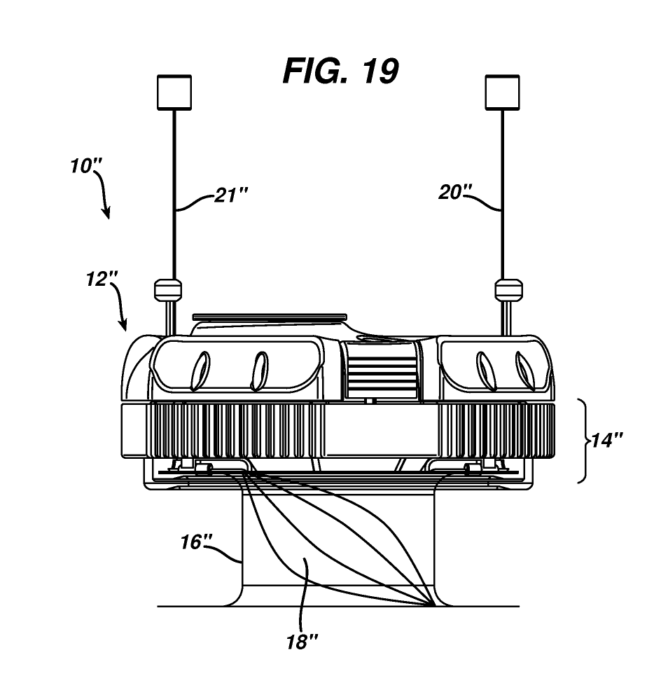

In another exemplary embodiment, as shown in FIG. 19, the access device 10″ can optionally include a second drawstring 21″ configured to contract or close a proximal circumference or perimeter of the pouch 18″. For example, the proximal circumference of the pouch 18″ can be cinched together by pulling the second drawstring 21″ proximally with respect to the access device 10″. To that end, the pouch 18″ can be releasably mated to the distal surface of the housing 12″, to the ring assembly 14″, and/or to the retractor 16″, for example using an adhesive or a friction fit, such that application of sufficient pulling force to the second drawstring 21″ is effective to peel away or otherwise separate the proximal circumference of the pouch 18″ from the access device 10″.

In some embodiments, a plurality of liner-type pouches 18′ or 18″ can be provided in a layered configuration within the retractor, each pouch having one or more corresponding drawstrings for effecting closure of one or more portions thereof (e.g., a proximal and a distal circumference thereof). In such embodiments, a plurality of specimens can be retrieved, each in separate pouches, without requiring removal and/or reinstallation of the access device. In embodiments in which the one or more pouches are mated to the housing, they can optionally be mated to the outer housing, such that the inner housing can still be rotated with respect to the pouch or pouches.

In use, the surgical access devices disclosed herein can provide access to a surgical site, such as a patient's body cavity, and can facilitate retrieval of one or more specimens therefrom. In one embodiment, a surgical access device can be installed or positioned in tissue while already fully assembled (e.g., in the configuration shown in FIGS. 1-3). Alternatively, the surgical access device can be positioned partially assembled in tissue and can then be fully assembled thereafter (e.g., while a portion of the access device is still positioned in the tissue). For example, in one embodiment the retractor can first be inserted into an opening in tissue with one or more drawstrings and/or support members already threaded through the retractor. Thereafter, the retractor ring can be assembled to the retractor and then the locking ring and housing can be coupled to the retractor ring, as described above. As the device is assembled, the drawstring(s) and/or support member(s) can be threaded up through the ring assembly and the housing. Once the access device is installed and/or assembled, insufflation fluid can be provided to the body cavity through an insufflation port formed in the access device.

In another exemplary embodiment, as explained above, the retractor can be installed with the pouch in a rolled or gathered configuration. In such embodiments, the support member can be inserted into the device after it is installed in the tissue wall. As the support member is installed, it can force the pouch to unroll or unfurl to a fully-deployed configuration.

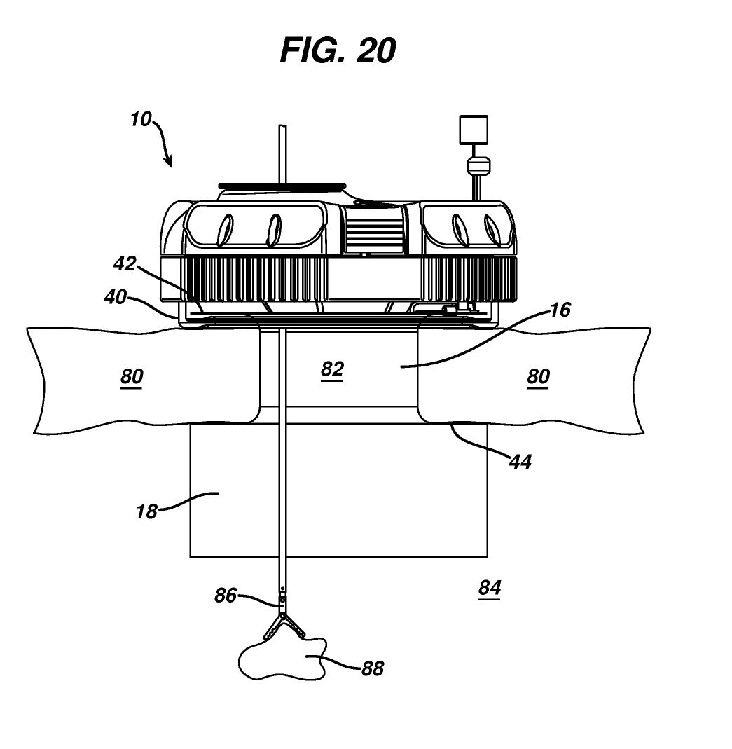

FIGS. 20-23 illustrate one exemplary method of retrieving a specimen using a surgical access device 10. As shown in FIG. 20, the retractor 16 of the access device 10 can be positioned within an opening in a tissue wall 80 such that the distal retractor flange 44 lies immediately beneath the tissue wall 80 and the proximal retractor flange 42 and the retractor ring 40 lie against an exterior surface of the tissue wall 80. The inner lumen 82 of the retractor 16 can form a pathway through the tissue wall 80 such that surgical instruments can be inserted from outside the body, through the access device 10, and into the interior of the body cavity 84. The elasticity of the tissue wall 80 can assist in the retention of the retractor 16 and the access device 10 in the opening or incision. The access device 10 can be placed in any opening within a patient's body, whether a natural orifice or an opening made by an incision. In one embodiment, the retractor 16 can be substantially flexible so that it can easily be maneuvered into and within tissue as needed. In other embodiments, the retractor 16 can be substantially rigid or substantially semi-rigid.

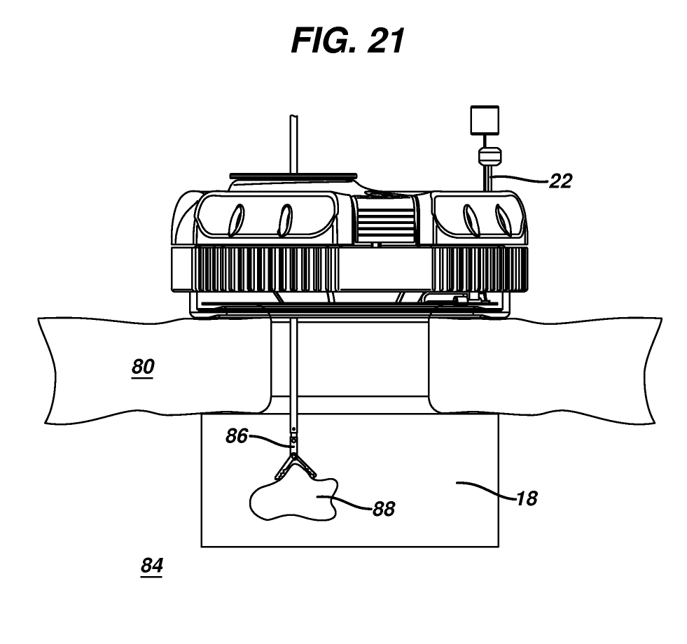

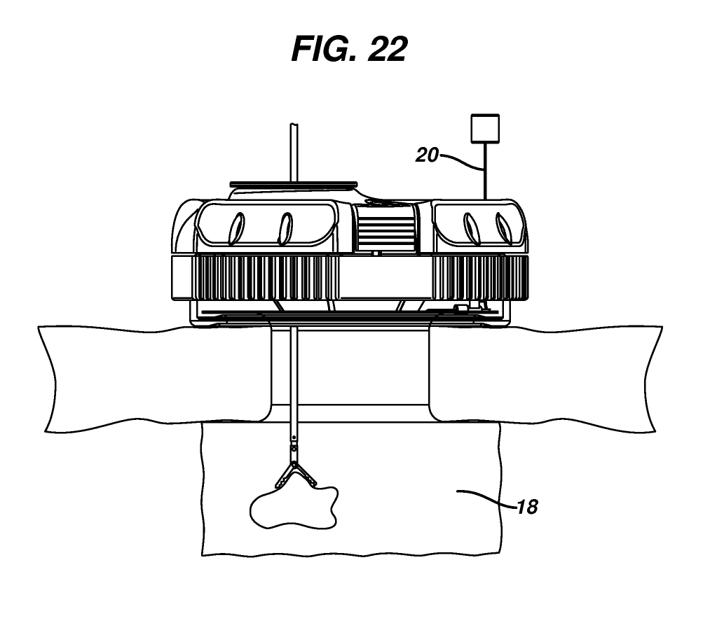

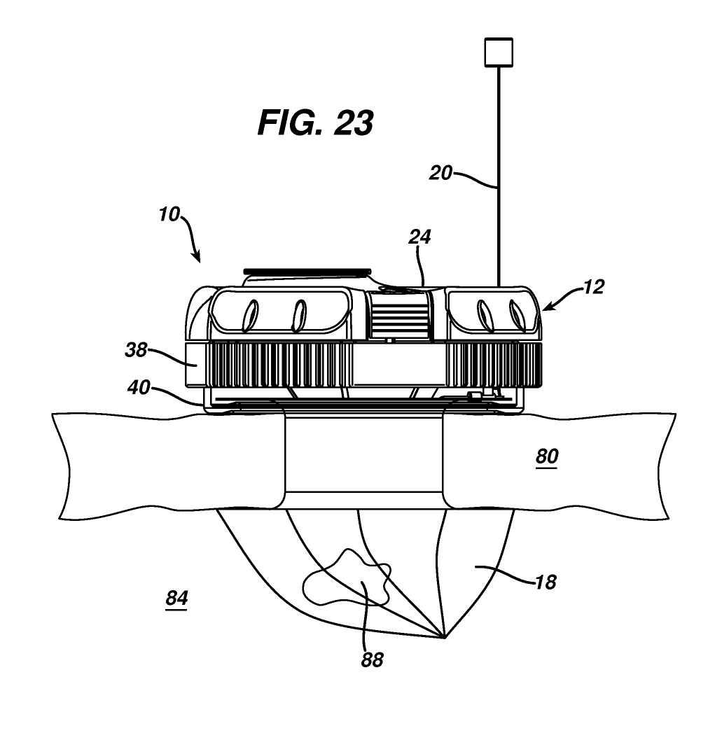

Once inserted through the tissue wall 80, one or more surgical instruments can be inserted through the access device 10 and into the body cavity 84 to retrieve a specimen 88 disposed therein. As shown in FIG. 20, laparoscopic graspers 86 can be used to grasp the specimen 88. As shown in FIG. 21, the graspers 86 can then be retracted proximally with respect to the access device 10, thereby drawing the specimen 88 into the specimen retrieval pouch 18. Once situated therein, the support member 22 can be removed from the access device 10 by pulling it proximally with respect thereto until the entire support member 22 has been removed from the device 10. As shown in FIG. 22, the pouch 18 can then be freely manipulated, for example by pulling the drawstring 20. As shown in FIG. 23, the drawstring 20 can be pulled proximally to contract and/or close a distal circumference of the pouch 18 around the specimen 88 or a portion thereof, thereby capturing the specimen 88 or a portion of the specimen 88 within the pouch 18. Once securely positioned and/or captured within the pouch 18, the specimen 88 can be released from the graspers 86 and the graspers 86 can be withdrawn from the device 10. One or more portions of the device (e.g., the inner housing 24 or the entire housing 12) can then be disassembled from the retractor 16 and the specimen retrieval pouch 18. For example, the locking ring 38 can be rotated with respect to the retractor ring 40 to allow removal of the entire housing 12 from the retractor 16. A user can then reach into the pouch 18, either by hand or using one or more surgical tools, and remove the specimen 88 from the body cavity 84. Alternatively, the retractor 16 and pouch 18 can be removed while the specimen 88 is still positioned therein, or the entire device 10 can be left in an assembled configuration and removed from the tissue wall 80 while the specimen 88 is disposed within the closed pouch 18. Each of these techniques can advantageously permit retrieval of a specimen that is too large or too delicate to be pulled through an instrument port or other working channel of the access device.

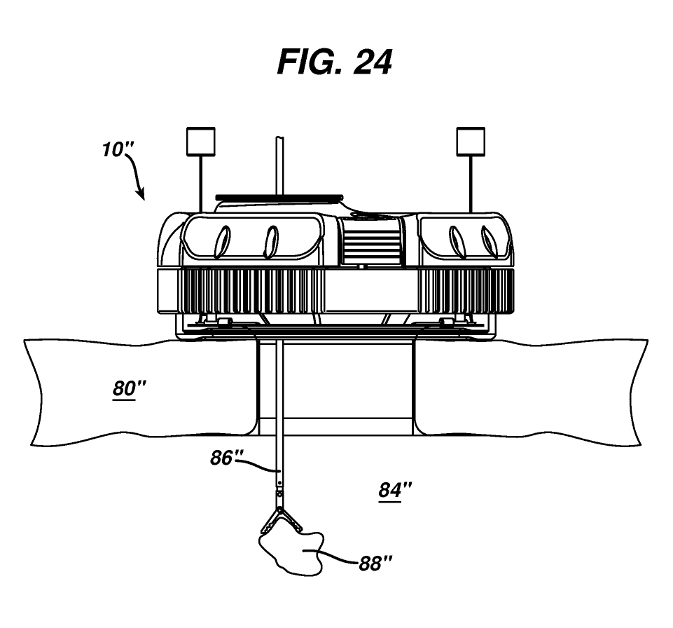

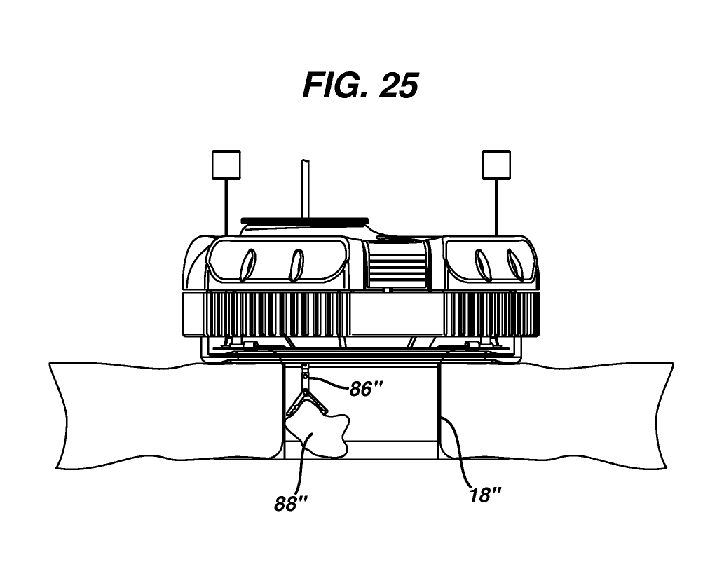

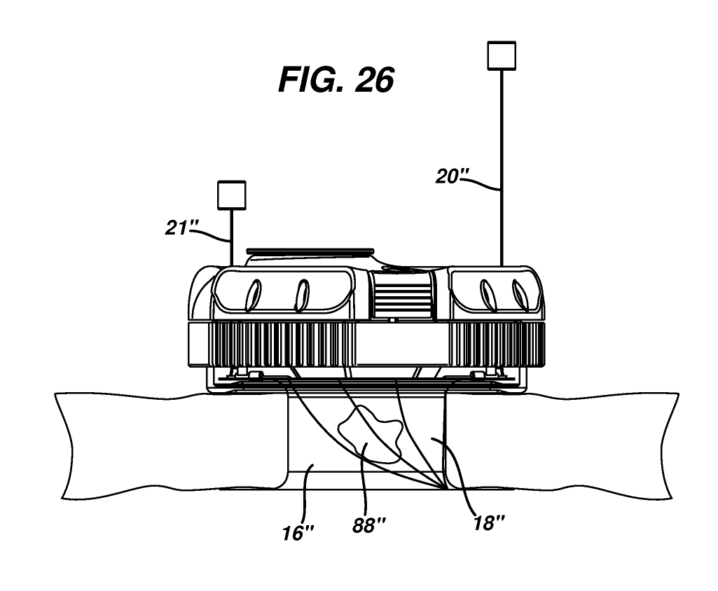

FIGS. 24-28 illustrate one exemplary method of using a surgical access device having a plurality of drawstrings to retrieve a specimen from within a body cavity. As shown in FIG. 24, a surgical access device 10″ can be inserted through an opening in a tissue wall 80″ as described above. A laparoscopic grasping tool 86″ can then be inserted through a working channel of the access device 10″ and used to grasp a specimen 88″ disposed within the body cavity 84″. The graspers 86″ can then be used to pull the specimen 88″ proximally until it is positioned within the specimen retrieval pouch 18″, as shown in FIG. 25. A first drawstring 20″ can then be pulled proximally as shown in FIG. 26 to contract and/or close a distal circumference of the pouch 18″ and capture the specimen 88″. In the illustrated embodiment, the pouch 18″ can be adhered to the inner surface of the retractor 16″ and pulling the drawstring 20″ can be effective to break the adhesive bond between the distal portion of the pouch 18″ and the distal portion of the retractor 16″. With the specimen 88″ secured and/or captured within the pouch 18″ and thus prevented from falling distally out of the access device 10″, the specimen 88″ can be released from the graspers 86″ and the graspers 86″ can be removed from the access device 10″.

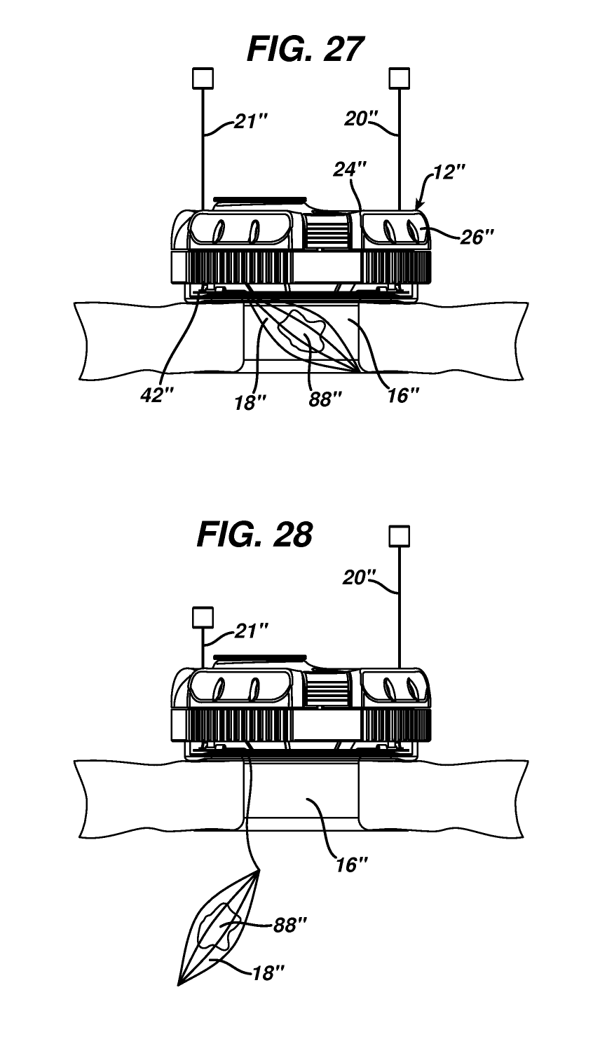

A second drawstring 21″ can then be pulled proximally, as shown in FIG. 27, to contract and/or close a proximal circumference of the pouch 18″, thereby fully enclosing the specimen 88″ within the pouch 18″. In one embodiment, the proximal circumference of the pouch can be adhered to the proximal flange 42″ of the retractor, and pulling the second drawstring 21″ can be effective to break the adhesive bond between the proximal portion of the pouch 18″ and the proximal retractor flange 42″. The pouch 18″, with the specimen 88″ enclosed within, can then be removed from the access device 10″. For example, the housing 12″ can be separated from the retractor 16″, advanced proximally along the drawstrings 20″, 21″, and temporarily positioned off to one side of the tissue opening. The pouch 18″ can then be grasped by the user and a knife or other cutting tool can be used to sever the drawstrings 20″, 21″ and permit removal of the entire pouch 18″ and specimen 88″ from the surgical site. Alternatively, graspers and cutting tools can be inserted through the access device 10″ to separate the pouch 18″ therefrom and then remove the pouch 18″, either through the outer housing 26″ after removing the inner housing 24″ or through a working channel formed in the inner housing 24″. It will be appreciated that a variety of other techniques for retrieving the pouch 18″ and specimen 88″ are possible in accordance with the present invention.

As shown in FIG. 28, the surgeon can optionally separate the specimen retrieval pouch 18″ from the access device and temporarily leave the pouch 18″ and specimen 88″ within the body cavity while other surgical activities are performed. For example, tension applied to the first and second drawstrings 20″, 21″ can be released, allowing the pouch 18″ to hang down from the distal end of the access device into the body cavity. This can advantageously allow the pouch to be moved out of the way so that other surgical procedures can be performed. By way of further example, a cutting tool (not shown) can be used to sever one or both of the drawstrings 20″, 21″ which can partially or completely separate the pouch 18″ from the access device.

As will be appreciated by those skilled in the art, any and all of the embodiments disclosed herein can be interchangeable with one another as needed. For example, a surgical access device kit could be provided and could include multiple housings, retractors, and/or specimen retrieval pouches, each having different sizes, configurations, etc. as needed in particular application.

In addition, the devices disclosed herein can be designed to be disposed of after a single use, or they can be designed to be used multiple times. In either case, however, the device can be reconditioned for reuse after at least one use. Reconditioning can include any combination of the steps of disassembly of the device, followed by cleaning or replacement of particular pieces, and subsequent reassembly. In particular, the device can be disassembled, and any number of the particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

Preferably, the invention described herein will be processed before surgery. First, a new or used instrument is obtained and if necessary cleaned. The instrument can then be sterilized. In one sterilization technique, the instrument is placed in a closed and sealed container, such as a plastic or TYVEK bag. The container and instrument are then placed in a field of radiation that can penetrate the container, such as gamma radiation, x-rays, or high-energy electrons. The radiation kills bacteria on the instrument and in the container. The sterilized instrument can then be stored in the sterile container. The sealed container keeps the instrument sterile until it is opened in the medical facility.

It is preferred that the device is sterilized. This can be done by any number of ways known to those skilled in the art including beta or gamma radiation, ethylene oxide, steam, and a liquid bath (e.g., cold soak).

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A surgical access device, comprising:
a housing having a proximal end and a distal end and at least one port extending therethrough, the at least one port being configured to receive a surgical instrument therethrough;
a specimen retrieval pouch coupled to the housing and configured to receive a specimen collected by a surgical instrument inserted through the at least one port; and
a first drawstring configured to contract a distal perimeter of the pouch.

2. The device of claim 1, wherein the housing comprises a retractor coupled to the distal end thereof, the retractor being configured to be disposed within tissue to form a pathway through the tissue and into a body cavity.

3. The device of claim 1, further comprising a second drawstring configured to contract a proximal perimeter of the pouch.

4. The device of claim 1, wherein the first drawstring extends from a distal end of the pouch and through a tunnel formed along an inner surface of the housing.

5. The device of claim 1, further comprising a resilient support member coupled to at least a portion of the distal perimeter of the pouch.

6. The device of claim 5, wherein the resilient support member is formed from a shape memory material.

7. The device of claim 5, wherein the resilient support member includes at least one sleeve configured to couple the first drawstring to the support member.

8. The device of claim 1, wherein the first drawstring extends around the entire distal perimeter of the pouch and includes a slipknot tied thereon.

9. The device of claim 8, wherein the slipknot is positioned within the housing, adjacent to a bearing surface of the housing configured to bear against the slipknot when the drawstring is translated proximally with respect to the housing.

10. The device of claim 1, wherein the pouch is substantially cylindrical.

11. A specimen retrieval system, comprising:
a surgical access device configured to be inserted through an opening in tissue;
a pouch coupled to the surgical access device; and
an actuator element extending through a sidewall of the surgical access device and coupled to the pouch such that tension applied to a proximal end of the actuator element is configured to contract a distal perimeter of the pouch.

12. The system of claim 11, wherein the actuator element comprises a length of suture extending around the distal perimeter of the pouch and having at least one slip knot tied thereon.

13. The system of claim 11, further comprising a support wire that runs alongside the actuator member.

14. The system of claim 11, wherein the surgical access device comprises a proximal housing having a tissue retractor coupled to a distal end thereof, and wherein the pouch is disposed within the tissue retractor.

15. A method for retrieving a specimen from a surgical site, comprising:
inserting an access device having a proximal end and a distal end through an opening in tissue to form a pathway through the tissue and into a body cavity;
pulling a specimen proximally into a pouch disposed within the access device; and
actuating a drawstring extending along a sidewall of the access device to contract a distal circumference of the pouch to retain at least a portion of the specimen within the pouch.

16. The method of claim 15, further comprising removing the access device and the specimen from the opening.

17. The method of claim 15, wherein pulling the specimen proximally comprises grasping the specimen with an instrument passed through the access device and retracting the instrument proximally with respect to the access device.

18. The method of claim 15, further comprising removing a support member from the access device before contracting the distal circumference of the pouch.

19. The method of claim 15, further comprising removing a housing portion of the access device having at least one seal disposed therein and then removing the specimen through the opening.

20. The method of claim 15, wherein actuating the drawstring comprises pulling a terminal end of the drawstring proximally with respect to the access device.
